(19) United States
(12) Patent Application Publication
(10) Pub. No.: US 2005/0042318 A1
(43) Pub. Date: Feb. 24, 2005

(54) Extracts from hop, methods for producing the same and their use

(76) Inventors: Clemens Erdelmeier, Karlsruhe (DE); Egon Koch, Karlsruhe (DE)

Correspondence Address: MCDONNELL BOEHNEN HULBERT & BERGHOFF LLP, 300 S. WACKER DRIVE, 32ND FLOOR, CHICAGO, IL, 60606, US

(21) Appl. No.: 10/486,390

(22) PCT Filed: Aug. 8, 2002
(86) PCT No.: PCT/EP02/08943

(30) Foreign Application Priority Data
Aug. 10, 2001 (DE) 101 39 479.9

(51) Int. Cl. A61K 35/78

(52) U.S. Cl. 424/778

(57) ABSTRACT

Novel extracts from hop with an increased content of prenylated chalcones and flavones, methods for producing the same, pharmaceutical preparations comprising such extracts of hop and use of these extracts of hop for the prophylaxis and treatment of pathological conditions caused by oestrogen deficiency or by dysregulations to sex-hormone-related metabolism are described.

The present invention relates to extracts from hop, methods for producing the same and their use for the prophylaxis and treatment of pathological conditions caused by oestrogen deficiency or by dysregulations to sex-hormone-related metabolism, in particular oestrogen metabolism.

The greatest importance of hop is still its use for producing beer. Due to its bittering and flavouring agents it is decisive for the taste of beer. Moreover, these agents have reached a certain relevance in conservating beer because of its antimicrobiotic characteristics.

The scientific knowledge in the field of hop in the early 80's resulted in a positive monograph from the agency E of the federal health authorities at that time (Bundesanzeiger dated Dec. 5, 1985 and Mar. 13, 1990, respectively). Thus, use of hop for the treatment of sleep disturbances, agitation and anxiety is basically permitted.

Already for a long time hop has a pharmaceutical relevance as a mild sedative in public medicine. Presumably α- and β-bitter acids which are sensitive to oxidations are responsible for this effect. In recent times it was shown that these ingredients also exhibit radical trap characteristics and lipid peroxidation inhibiting properties (M. Tagashira et al., Biosci. Biotech. Biochem. 59, 740-742 (1995)). Furthermore, pharmaceutical compositions for the treatment of osteoporosis are described in the European patent application 0 677 289 A2 which contain compounds from the group of α-bitter acids and α-iso-bitter acids.

In the last years also the phenolic ingredients of hop were increasingly examined beside α- and β-bitter acids (J. H\check{o}lzl, Zeitschrift für Phytotherapie 13, 155-161 (1992)) and beside the longer known Xanthohumol 1 further flavone-type compounds were found in hop plants (J. F. Stevens et al., Phytochemistry 44, 1575-1585 (1997), J. F. Stevens et al., J. Chromat. A 832 (1-2), 97-107 (1999)). These were primarily isoprenylated flavonoides, such as for example 6- or 8-prenylnaringenine 2 and 3 and isoxanthohumol 4. Stevens et al. (Phytochemistry 53, 759-775 (2000)) also examined the chemotaxonomy of species of hop and taxa of hop.

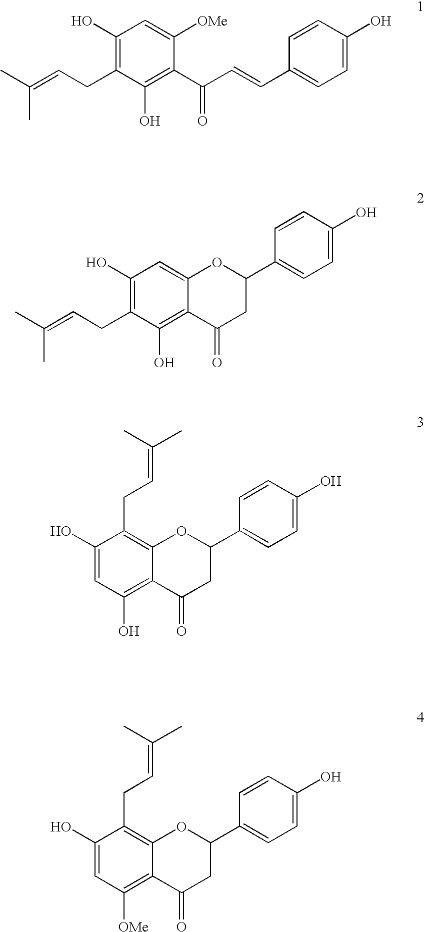

Again and again it was observed that menstrual disorders appeared in women that picked hop, which were traced back to oestrogenic substances in hop, but these effects could not clearly be associated with one or more ingredients. In the meantime this oestrogenic activity of hop could be confirmed. Thus, it was demonstrated that 8-prenylnaringenine 3 is essentially responsible for these effects (S. R. Milligan et al., J. Clin. Endocrinol. Metab. 84, 2249-2252 (1999)). The oestrogenic activity in vitro of this compound appeared from its relative binding affinity to oestrogenic receptors and was particularly tested by stimulating alkaline phosphatase in Ishikawa-Var-I-cells. Thus, it was demonstrated that 8-prenylnaringenine was significantly more active than the phytooestrogenes known so far such as coumestrol, genistein or daidzein, and produced only a slightly weaker effect than 17β oestradiol. Milligan et al. (J. Endocrin. Metabol. 85, 4912-4915 (2000)) also reported about the bonding of different phenolic ingredients of hop to a human oestrogenic receptor expressed in yeast cells. Thereby, again 8-prenylnaringenine exhibited the most powerful oestrogenic activity. 6-Prenylnaringenine, 6,8-diprenylnaringenine and 8-geranylnaringenine exhibited weaker oestrogenic properties. Miyamoto et al. (Planta Med. 64, 516-519 (1998)) could demonstrate that 8-prenylnaringenine normalises the weight of the uterus and osseous density at ovariectomised rats. Moreover, the oestrogen agonistic activity of a series of 8-prenylated flavone derivatives including 8-prenylnaringenine is described in JP 08 165238 (ref. CA 125:158632).

Recent studies could demonstrate that some flavonoides of hop, particularly xanthohumol 1, can influence the cell metabolism. They are capable of positively influence enzymatic reactions that play an important role in the development of tumour cells. For this reason these compounds can be considered as preventive agents against cancer (conference of the German Society of Hop Research, present German Society of Hop Research, present knowledge concerning the hop ingredient xanthohumol, Mar. 24, 1998, Aschheim). Miranda et al. (Food Chem. Tox. 37(4), 271-285 (1999)) reported strong antiproliferative activity of xanthohumol 1 and isoxanthohumol 4 in human breast cancer cells MCF-7, colon cancer cell lines HAT-29 and ovarian cancer cell lines A-2780.

It could further be demonstrated that xanthohumol 1 has an inhibiting effect on osteolysis. Its use as a therapeutic agent against osteoporosis is described in the European patent EP 0 679 393 B1. Although the inventors postulate oestrogenic properties of xanthohumol, these properties are not demonstrated. Contrarily, S. R. Milligan et al. (Pharm. Pharmacol. Lett. 7, 83-86 (1997)) clearly excluded that the osteoporosis inhibiting activity of xanthohumol is based on an oestrogenic effect because the corresponding activities could not be detected for both the human endometrial carcinoma cell line Ishikawa and in an yeast reporter gene assay (S. R. Milligan et al., J. Clin. Endocrinol. Metabol. 84, 2249-2252 (1999)). Contrarily to these examinations the present invention demonstrates that xanthohumol 1 and isoxanthohumol 4 having a comparable activity bind to the oestrogenic receptors alpha and beta.

Kumai and Okamoto (Toxicology Letters 21, 203-207 (1984)) reported about high-molecular carbohydrate fractions from merely aqueous extracts of hop reducing the ovarian weight of young rats pretreated with PMS gonadotropin. Okamoto and Kumai (Acta Endocrinologica 127, 371-377 (1992)) confirmed these results on the basis of the observation of reduced blood levels of 17β oestradiol and LH caused by administering the merely aqueous extract from hop.

An extract from hop having an increased content of xanthohumol is described in the German patent application DE 199 39 350 A1. This extract is to be added to beer and to soft drinks containing fruit juice. There is nothing known about the presence of prenylated naringenines in this extract. According to an embodiment xanthohumol is extracted from the hop with 50% by weight ethanol. However, this does not result in an ideal extraction of xanthohumol because a high degree of transition into the extract cannot be obtained, until ethanol having a high content (>80% by weight) is used.

A method for the isolation of oestrogenically active compounds from hop is claimed in WO 83/00701 A1, characterised in that a carbon dioxide extract from hop is produced first adding water as an entraining agent and subsequently the oestrogenically active compounds are obtained therefrom by means of an extraction using ether or chromatographic methods. Moreover, the use of these compounds as an additive for animal feed, for cosmetic agents or as a bathing additive is claimed. There do not appear any particulars about the type of these oestrogenically active compounds.

A method for the isolation of stable additives for brewing beer is claimed in WO 01/30961 A1, characterised in that the residue of the hop draff from the carbon dioxide extraction is extracted with a polar solvent, preferably hot water and the extract is subsequently acidified, washed with a nonpolar solvent, preferably hexane, and—optionally after drying—used as a brewing additive. The remaining draff is discarded.

The object underlying the present invention is to provide plant extracts that are suitable for the production of pharmaceuticals for the prophylaxis and treatment of pathological conditions that are caused by an oestrogen deficiency or by dysregulations to sex-related-homone metabolism, in particular oestrogen metabolism.

A further object of the present invention is to provide a method for producing such extracts and pharmaceutical preparations comprising the same suitable for the treatment of the pathological conditions mentioned above.

According to the present invention these objects are solved by the extract from hop according to claims 1 and 2, the methods according to claims 3 to 12, the pharmaceutical preparation according to claim 13 and the use of the extracts or the pharmaceutical preparation according to claims 14 to 16.

The present invention relies inter alia on the surprising observation that after removing lipophilic and hydrophilic fibres extracts are obtained from the hop drug still containing phloroglucinol-type bitter acids of hop and simultaneously containing free and/or bonded chalcones and flavones such as xanthohumol, isoxanthohumol as well as 6- and 8-prenylnaringenine in an enriched form. The fact that the content of 6- and 8-prenylnaringenine depends on the temperature of the preextraction with water (cf. Example 3) and that it can be enhanced by a factor of up to about 2 is particularly surprising.

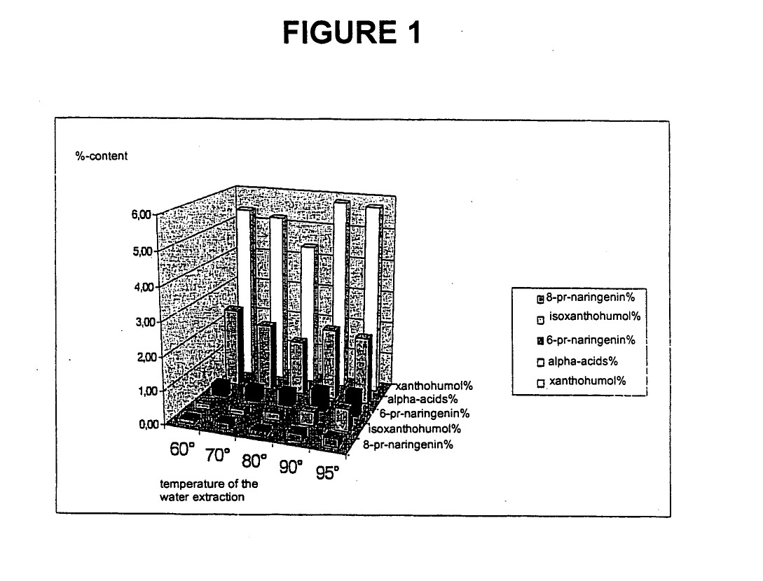

FIG. 1 demonstrates the dependence of the concentration of the analysed ingredients on the temperature of the preextraction with water.

Such an extract can be obtained by one or more extractions using a C₅-C₇-alkane or supercritical CO₂ (step of removing fat), subsequent extraction of the remaining drug residue using water and followed by extraction of the still remaining drug residue using a solvent of medium polarity selected from the group consisting of alcohols, aqueous alcohols, ketones, aqueous ketones, esters and optionally subsequent liquid-liquid distribution. Surprisingly the transition of the bitter acids of hop into the lipophilic extract does not occur completely but only in parts. On the other hand, the chalcones and flavones remain nearly completely in the drug residue during the extraction using water. Thereby, an extract from hop is obtainable containing all of the pharmacologically relevant ingredients (bitter acids, chalcones, flavones) in a balanced ratio. Due to this advantageous composition using several therapeutic principles this extract is ideally applicable against pathological conditions caused by oestrogen deficiency or by other hormone-related dysregulations.

The extracts from hop according to the present invention are suitable for the prophylaxis and treatment of climacteric-related complaints or post menopause-related complaints in women, the symptoms comprising inter alia hot flushes, depression, anxiety, mental confusion, insomnia and post menopause-related serious health problems such as osteoporosis, diseases of the cardiovascular system, cerebral infarction (strokes), dementia and tumour diseases. Other diseases which are based on a dysregulation of the sex-hormone-related metabolism and which can be treated using the extract according to the present invention are for example amenorrhea, anovulatory cycles, menometrorrhagia, premenstrual complaints and postpartal depressions. Likewise these extracts can be used for the treatment of sex-hormone-dependent diseases in men such as for example benign prostatic hypertrophy or carcinoma of the prostate.

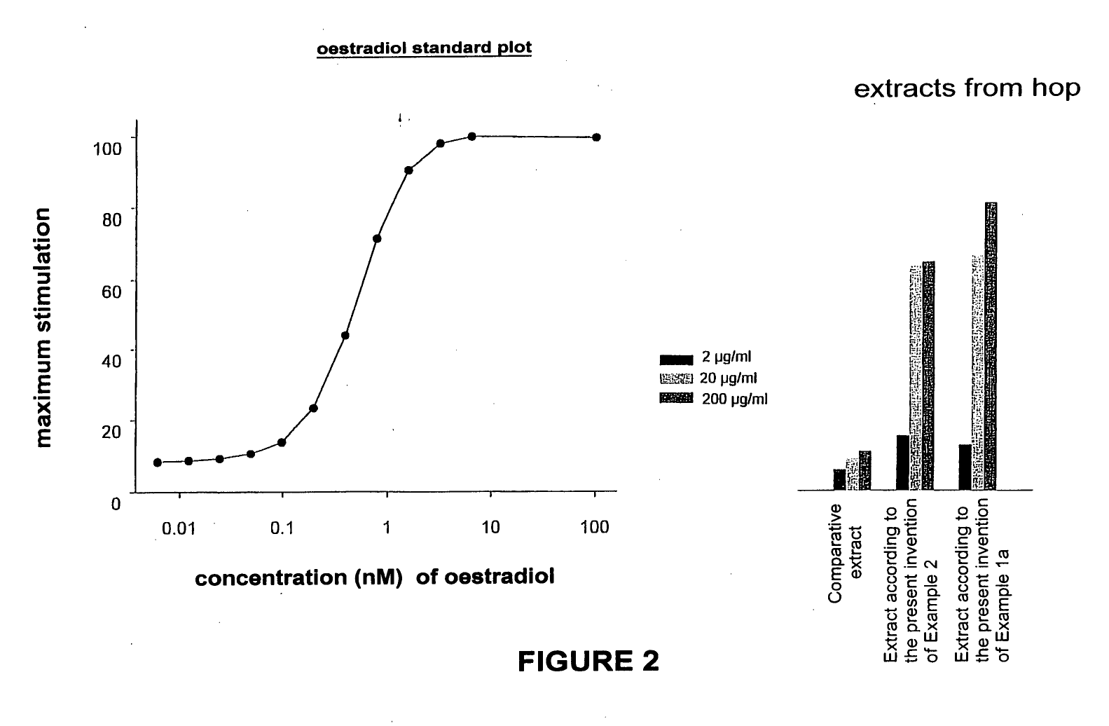

The surprisingly high oestrogenic activity of the extracts from hop according to the present invention were detected both using a competitive receptor binding assay for the human oestrogen receptors alpha and beta and in a recombinant yeast assay compared to the activity of 17β oestradiol. Contrarily, conventional standard extracts of hop having the same dosage exhibit an essentially lower activity or no activity at all. FIG. 2 illustrates the activity of the comparative extract and of two extracts of hop according to the present invention in a yeast reporter gene assay.

According to the present invention, an extract from hop having an increased content of free and/or bonded chalcones and flavones, in particular 6- and 8-prenylnaringenine, xanthohumol and isoxanthohumol compared to conventional, in particular aqueous alcoholic extracts is provided, which simultaneously further contains α and possibly β bitter acids (humulone and lupolone and derivatives thereof, respectively).

Furthermore, according to the present invention a method for the preparation of these extracts from hop is provided, comprising the steps of:
(a) one or more extractions of a hop drug using a C₅-C₇-alkane or supercritical CO₂ and separating the drug residue from the extraction solution;
(b) one or more extractions of the drug residue obtained in step (a) using water and separating the drug residue;
(c) one or more extractions of the drug residue obtained in step (b) using a solvent selected from the group consisting of alcohols, aqueous alcohols, ketones, aqueous ketones and esters and filtration of the extraction solution obtained; and
(d) removing the solvent from the combined extraction solutions obtained in step (c) and drying of the residue obtained.

The drug-to-solvent ratio for every extraction step is in the range of about 1:7 to about 1:12.

The extraction using a C₅-C₇-alkane or supercritical CO₂ in step (a) is preferably carried out once, twice or three times, particularly three times.

The extraction using supercritical CO₂ is particularly preferred.

The C₅-C₇-alkane used in step (a) is preferably a C₅-C₇-n-alkane selected from the group consisting of n-pentane, n-hexane, and n-heptane, with n-heptane being particularly preferred.

The extraction according to step (b) is preferably carried out at a temperature between 60 and 95° C., preferably at 90° C., wherein the duration of the extraction can take one or more hours.

The solvent used in step (c) is preferably selected from the group consisting of ethanol, aqueous ethanol, methanol, aqueous methanol, acetone, aqueous acetone and ethyl acetate, with 80 to 96% (w/w) ethanol, 74 to 99% (w/w) methanol and 60 to 99% (w/w) acetone being preferred, respectively, and 92% (w/w) ethanol being particularly preferred.

The (dry) extract from hop according to the present invention is characterised by having a content of a bitter acids of at least 0.5%, preferably at least 0.8% and particularly preferred 1%, a content of xanthohumol of at least 2%, preferably at least 3% and particularly preferred 4%, and a content of prenylated flavones of at least 0.5%, preferably at least 0.7%. The prenylated flavones preferably comprise 6-prenylnaringenin, 8-prenylnaringenin and isoxanthohumol. For the purpose of the present invention xanthohumol is not among the prenylated flavones. Percentage details refer to the weight of the dry extract from hop.

The extracts obtained can be processed together with conventional pharmaceutically acceptable additives to pharmaceutical preparations such as capsules, film tablets or coated tablets. Fillings, bonding agents, disintegrants, lubricants and coatings for film tablets and coated tablets, as well as oils and fats as filling agents for gelatine capsules, can be used as pharmaceutical additives.

The extracts according to the present invention can be used for the prophylaxis and treatment of pathological conditions, caused by oestrogen deficiency or by other hormone-related dysregulations, such as particularly climacteric complaints, sex-hormone-dependent cancer diseases, benign prostatic hypertrophy, osteoporosis, Alzheimer's disease and diseases of the cardiovascular system. In case of sex-hormone-dependent cancer diseases the extracts according to the present invention can be particularly used for the prophylaxis and the treatment of breast cancer, carcinoma of the uterus and prostatic carcinoma.

The dosage of the extracts according to the present invention is in the range from 0.005 g to 2 g extract 1 to 4 times per day, preferably in the range from 0.02 g to 1 g 1 to 2 times per day. In an individual case, the dosage depends on the clinical picture and on the individual circumstances of the patient and can be adjusted to the respective requirements by the attending practitioner.

The examples given below illustrate the invention and should not be considered to limit the invention. All percentage details refer to the weight, unless specified otherwise.

### COMPARATIVE EXAMPLE

### Production of an Extract Using 96% (w/w) Ethanol Without Prior Degreasing

50 g of drug of hop (sort “Hallertauer Magnum”) were mixed with 500 g 96% (w/w) ethanol and reduced to small peaces using an ultraturrax. The extraction was carried out for 1 hour at 60° C. Then the extract was filtered using a filter type Seitz 1500. The drug was extracted further 2 times in the same way. The combined extraction solutions were set free from the ethanol on a rotary evaporator and dried overnight at 50° C. in a vacuum drying cabinet. The content of characteristic ingredients was determined from the dry matter using the following HPLC method. This HPLC method is also applied for the determination of ingredients in the other examples.

| | |
|---|---|
| Column | LiChrospher |
| | 100 5 μm. |
| | 250 × 4 mm |
| Eluant | A: 1000 ml bidest. |
| | water/3 ml |
| | phosphoric acid |
| | (85%)/2 ml |
| | triethylamine |
| | B: 1000 ml |
| | acetonitrile/3 ml |
| | phosphoric acid |
| | (85%)/2 ml |
| | triethylamine/60 ml |
| | bidest water |
| Gradient | from 40% B to 70% B |
| | within 30 min; |
| | from 70% B to |
| | 100% B within 10 min |
| Flow | 1.2 ml/min |
| Detection | diode array |
| Yield (96% (w/w) | 18.38 g => 36.8% |
| ethanol extract): | |
| HPLC content of | 19.8% |
| α bitter acid | |
| of hop: | |
| HPLC content of | 4.2% |
| β bitter | |
| acid of hop: | |
| HPLC content | 1.3% |
| of xanthohumol: | |
| HPLC content of | below |
| 6- and 8-prenylnaringenines | the detection |
| as well as | limit (<0.01%) |
| isoxanthohumol: | |

### Example 1a

### Production of an Extract from Hop (Extraction Using CO₂ and Subsequent Preextraction at 90° C. Using Water)

Sequential extraction using supercritical CO₂, water and 92% (w/w) ethanol:

80.6 g of a drug of hop (sort “Hallertauer Magnum”) which had been preextracted using supercritical CO₂ (conditions: milling to a grain size of 10 mm, extraction using CO₂ at 250 bar/50° C., separating the extract with a yield of 30%) were initially extracted using 960 g of water for 5 minutes on an ultraturrax, then extracted for 1 hour at 90° C. while being stirred. Subsequently the water extract was filtered over a Seitz Supra Filter 1500. Then the still somewhat wet drug residue was extracted twice using 800 g of 92% (w/w) ethanol for 1 hour at 60° C., respectively. Subsequently it was filtered over a Seitz Supra 1500 and the extraction solution was set free from ethanol on a rotary evaporator having a temperature of the water bath of 55 to 65° C. and dried at 60° C. in the drying cabinet.

| Yields: | |
|---|---|
| Residue from water extraction: | 18.96 g (23.5%) |
| Residue from the extraction using 92% (w/w) EtOH: | 9.83 g (12.2%) |
| HPLC contents (based on the | |
| extract using 92% (w/w) EtOH): | |
| HPLC contents of α bitter acids of hop: | 2% |
| HPLC contents of β bitter acids of hop: | 0.5% |
| HPLC content of xanthohumol | 5.83% |
| HPLC content of 6-prenylnaringenine | 0.63% |
| HPLC content of 8-prenylnaringenine | 0.21% |
| HPLC content of isoxanthohumol | 0.42% |

### Example 1b

### Production of an Extract from Hop (Extraction Using CO₂ and Subsequent Preextraction at 90° C. Using Water)

Sequential extraction using supercritical CO₂, water and 92% (w/w) ethanol:

504.26 g of a drug of hop (sort “Hallertauer Magnum”) which had been initially preextracted using supercritical CO₂ (conditions: milling to a grain size of 10 mm, extraction using CO₂ at 250 bar/50° C., separating the extract with a yield of 30%) were extracted using 6 kg of water initially for 5 minutes on an ultraturrax, then extracted for 1 hour at 90° C. while being stirred. Then the water extract was filtered over a Seitz Supra Filter 1500. Then the still somewhat wet drug residue was extracted twice using 5 kg of 92% (w/w) ethanol for 1 hour at 60° C., respectively. It was filtered over a Seitz Supra 1500 and the extraction solution was set free from ethanol on a rotary evaporator having a temperature of the water bath of 55 to 65° C. and dried at 60° C. in a drying cabinet.

| Yields: | |
|---|---|
| Residue from the extraction using water: | 105.9 g (21%) |
| Residue from the extraction using 92% (w/w) EtOH: | 69.37 g (13.8%) |
| HPLC contents (based on the | |
| extract using 92% (w/w) EtOH): | |
| HPLC content of α bitter acids of hop: | 1% |
| HPLC content of β bitter acids of hop: | 0.5% |
| HPLC content of xanthohumol | 4.41% |
| HPLC content of 6-prenylnaringenine | 0.49% |
| HPLC content of 8-prenylnaringenine | 0.15% |
| HPLC content of isoxanthohumol | 0.6% |

### Example 2

### Production of an Extract from Hop (Extraction Using CO₂ and Subseqdently Preextraction at 60° C. Using Water)

Sequential extraction using supercritical CO₂, water and 92% (w/w) ethanol:

80.36 g of a drug of hop (sort “Hallertauer Magnum”) which had been preextracted using supercritical CO₂ (conditions: milling to a grain size of 10 mm, extraction using CO₂ at 250 bar/50° C., separating the extract with a yield of 30%) were initially extracted using 964 g of water for 5 minutes on an ultraturrax, then extracted for 1 hour at 60° C. while being stirred. Then the water extract was filtered over a Seitz Supra Filter 1500. Then the still somewhat wet drug residue was extracted twice using 800 g of 92% (w/w) ethanol initially for 5 minutes on an ultraturrax, respectively, subsequently extracted for 1 hour at 60° C. while being stirred, respectively. Then it was filtered over a Seitz Supra 1500 and the extraction solution was set free from ethanol on a rotary evaporator having a temperature of the water bath of 55 to 65° C. and dried at 60° C. in the drying cabinet.

| Yields: | |
|---|---|
| Residue from the extraction using water: | 17.91 g (22%) |
| Residue from the extraction using 92% (w/w) EtOH: | 9.95 g (12.4%) |
| HPLC contents (based on the | |
| extract using 92% (w/w) EtOH): | |
| HPLC content of α bitter acids of hop: | 1.58% |
| HPLC content of β bitter acids of hop: | 0% |
| HPLC content of xanthohumol | 6.1% |
| HPLC content of 6-prenylnaringenine | 0.4% |
| HPLC content of 8-prenylnaringenine | 0.09% |
| HPLC content of isoxanthohumol | 0.21% |

### Example 3

### Production of an Extract from Hop (Extraction Using n-Heptane and Subsequently Water at 90° C.)

247.6 g of a drug of hop (sort “Hallertauer Magnum”) were extracted using 7 times its weight made up of n-heptane initially for 5 minutes on an ultraturrax, then extracted for 1 hour while being stirred. After filtering the heptane extraction solution over a Seitz Supra 1500 it was extracted for a second time in the same way. Afterwards the obtained drug residue was set free from heptane in a vacuum drying cabinet. The dry drug residue (205 g) was then mixed with 12 times its weight made up of water and maintained for 1 hour at 90° C. Subsequently it was filtered again and the still somewhat wet drug residue was extracted twice at 60° C. using 10 times its weight made up of 92% (w/w) ethanol while being stirred. It was then filtered over a Seitz Supra 1500 and the extraction solution was set free from ethanol on a rotary evaporator having a temperature of the water bath of 55 to 65° C. and dried at 60° C. in a drying cabinet.

| Yields: | |
|---|---|
| extract using heptane: | 26.4 g (10.7%) |
| extract using water: | 41.1 g (16.6%) |
| extract using 92% (w/w) ethanol: | 52.0 g (21.0%) |
| HPLC contents (based on the extract | |
| using 92% (w/w) ethanol): | |
| α bitter acids: | 0.86% |
| β bitter acids: | 0.05% |
| xanthohumol: | 3.3% |
| 6-prenylnaringenine | 0.45% |
| 8-prenylnaringenine | 0.13% |
| isoxanthohumol | 0.25% |

### Example 4

### Dependence of the Content of 6-Prenylnaringenin, 8-Prenylnaringenin, and Isoxanthohumol on the Temperature of the Preextraction Using Water

Extraction: About 80 g of a drug of hop being preextracled using CO₂ were extracted using 12 times its weight made up of water initially for 5 minutes on a ultraturrax, then extracted for 1 hour at 60, 70, 80, 90, and 95° C. while being stirred. Then the extract using water was filtered over a Seitz Supra Filter 1500. Subsequently the still somewhat wet drug residue was extracted twice using 800 g of 92% (w/w) ethanol initially for 5 minutes on an ultraturrax, respectively, then extracted for 1 hour at 60° C. while being stirred. It was then filtered over a Seitz Supra 1500 and the extraction solution was set free from ethanol on a rotary evaporator having a temperature of the bath of 55 to 65° and dried at 60° C. in a drying cabinet.

The results being graphically represented in FIG. 1 exhibit a significant dependence of the concentration of the analysed prenylated ingredients on the temperature of the preextraction using water.

### Example 5

### Testing the Extracts from Hop for Oestrogenic Activity

The testing of individual ingredients of extracts, a comparative extract and an extract according to the present invention for interactions with the human oestrogenic receptor alpha (ER-α) and beta (ER-β), respectively, a competitive receptor binding assay was carried out. Thereby, a radioactively labelled oestradiol was initially bonded to the human oestrogen receptor and then treated with the test substance to be examined. A portion of the labelled oestradiol which corresponds to the oestrogenic potency of the sample is thus displaced. Excessive oestradiol is washed out after bonding of the complex to hydroxylapatite. The oestrogenic receptors ER-α and ER-β were commercially available as recombinant human receptors. The test preparations consisted of 1000 μl TEDG buffer (10 mM tris, 1,5 mM EDTA, 10% glycerol, pH 7.5), 5 μl receptor (200 nM), 10 μl 3H-oestradiol and 10 μl ethanol (control value), 10 μl diethylstiloestrol (100 mM, positive control) or 10 μl extract or ingredient of the extract, respectively. The preparations were carefully mixed and incubated for about 16 hours at room temperature in the dark. After incubation 250 μl hydroxylapatite (HAP) were added to absorb the proteins. During a period of incubation of 15 minutes the preparations were manually mixed with a time-lag of 5 minutes, respectively. The precipitate is separated by centrifugation at 10,000 rpm for a few seconds and the supernatant liquid is separated by pipetting. The pellet is washed three times with 1000 μl TEDG buffer, respectively, 1000 μl ethanol are added for the measurement, the mixture is slurried and transferred to a scintillation vial. After adding 9 ml scintillator liquid (Ready Safe, Beckmann) a measurement over the complete ³H window is carried out using a Beckmann Beta-Counter.

The characterisation of the bonding capacities of the test substances results from the determination of the ED₅₀ values taken from the dosis-effect-plots of the displacement of oestradiol. The results are reported in Table 1 and demonstrate potent interactions with both oestrogen receptors for all ingredients examined. Surprisingly the extract according to the present invention was proved to be essentially more effective than it had to be expected due the activities of the single ingredients. In contrast, the comparative extract exhibited an activity to both receptors which was at least 10 times smaller than that of the extract according to the present invention.

Table 1: Bonding of ingredients of the extracts, an extract according to the present invention and a comparative extract to the human oestrogen receptor-alpha (ER-α) and oestrogen receptor-beta (ER-β), respectively.

TABLE 1
Bonding of ingredients of the extracts, an extract according to the present
invention and a comparative extract to the human oestrogen receptor-alpha
(ER-α) and oestrogen recector-beta (ER-β), respectively.

| | ED₅₀ [pg/ml] | | relative potency | | |
|---|---|---|---|---|---|
| Substance | ER-α | ER-β3 | ER-α | ER-β | $\frac{relative potency ER \text{-} α}{relative potency ER \text{-} β}$ |
| 17β-Oestradiol | 507 | 400 | 1 | 1 | 1 |
| 8-Prenylnaringenine | 4.6 × 10⁴ | 1.0 × 10⁵ | 1.1 × 10⁻² | 4.0 × 10⁻³ | 2.72 |
| 6-Prenylnaringenine | 1.6 × 10⁶ | 4.6 × 10⁵ | 3.2 × 10⁻⁴ | 8.7 × 10⁻⁴ | 0.37 |
| Isoxanthohumol | 2.0 × 10⁶ | 8.5 × 10⁵ | 2.5 × 10⁻⁴ | 4.7 × 10⁻⁴ | 0.54 |
| Xanthohumol | 2.0 × 10⁶ | 1.2 × 10⁶ | 2.5 × 10⁻⁴ | 3.3 × 10⁻⁴ | 0.78 |
| Extract according to | 3.9 × 10⁵ | 2.7 × 10⁵ | 1.3 × 10⁻³ | 1.5 × 10⁻³ | 0.87 |
| the present invention | | | | | |
| Comparative extract | 4.0 × 10⁶ | 4.3 × 10⁶ | 1.3 × 10⁻⁴ | 9.4 × 10⁻⁵ | 1.33 |

Moreover, testing of extracts for oestrogenic properties was carried out with a reporter gene assay using yeast cells (saccharomyces). The cells are stably transfected with the human α-oestrogen receptor and an expression plasmid containing an oestrogen response element and the gene for the enzyme β-galactosidase. All samples were dissolved in DMSO at a concentration of 20 mg/ml, and were given undiluted or after diluting with DMSO at a ratio of 1/10, 1/100 or 1/1000 at a volume of 1 μl to 100 μl culture medium in 96-well flat-bottom micro-titre dishes. Next, 100 μl yeast suspension and the chromogenic substrate chlorophenol red-β-D-galactopyranoside were added. Control wells were provided on every dish, which were filled with either the culture medium or the solvent alone, or which contain the standard concentration of 17β oestradiol. The yeast cells were incubated for 72 hours at 32° C., after which absorption of the medium was measured at 540 nm in a micro-titre dish photometer. The samples were partially checked twice.

Results:

| Sample | activity |
|---|---|
| Extract using 96% (w/w) ethanol | inactive |
| according to comparative example | |
| Extract using 92% (w/w) ethanol | active |
| according to Example 1a | |
| Extract using 92% (w/w) ethanol | active |
| according to Example 2 | |

The results of the assays are depicted in FIG. 2. Hereby, those extracts are categorized as “active”, whose activity is significantly above the background values (corresponding to about 10% of the maximum stimulation) compared to the 17β oestradiol standard plot.

1. Method for obtaining an extract from hop, comprising the steps of:
(a) one or more extractions of a drug from hop with a C₅-C₇-alkane or supercritical CO₂ and separating the drug residue from the solution;
(b) one or more extractions of the drug residue obtained in step (a) with water at a temperature in the range of 60 to 95% and separating the drug residue;
(c) one or more extractions of the drug residue obtained in step (b) with 80-96% (w/w) ethanol and filtration of the obtained extraction solution; and
(d) removing the solvent from the combined extraction solutions obtained in step (c) and drying of the obtained residue.

2. Method according to claim 1, wherein the extraction in step (a) is carried out once, twice, or three times.

3. Method according to any one of claims 1 or 2, wherein the solvent in step (a) is selected from the group consisting of n-pentane, n-hexane, and n-heptane.

4. Method according to claim 3, wherein the solvent in step (a) is n-heptane.

5. Method according to anyone of claims 1 to 4, wherein the extraction in step (b) is carried out at about 90° C.

6. Method according to anyone of claims 1 to 5, wherein the solvent in step (c) is 92% (w/w) ethanol.

7. Method according to claim 1, wherein the solvent in step (a) is n-heptane and the solvent in step (c) is 92% (w/w) ethanol.

8. Method according to claim 1, wherein the solvent in step (a) is supercritical CO₂ and the solvent in step (c) is 92% (w/w) ethanol.

9. Extract from hop obtainable according to anyone of claims 1 to 8, characterized by having a content of a bitter acids of at least 0.5%, of xanthohumol of at least 2%, and of prenylated flavones selected from the group comprising 6-prenylnaringenin, 8-prenylnaringenin and isoxanthohumol of at least 0.5%.

10. Extract from hop according to claim 9, characterized by having a content of a bitter acids of at least 0.8%, of xanthohumol of at least 3%, and of prenylated flavones selected from the group comprising 6-prenylnaringenin, 8-prenylnaringenin, and isoxanthohumol of at least 0.7%.

11. Pharmaceutical preparation, comprising an extract from hop characterized by having a content of a bitter acids of at least 0.5%, of xanthohumol of at least 2%, and of prenylated flavones selected from the group comprising 6-prenylnaringenin, 8-prenylnaringenin and isoxanthohumol of at least 0.5% and conventional pharmaceutically acceptable additives.

12. Pharmaceutical preparation according to claim 11, comprising an extract from hop, characterized by having a content of a bitter acids of at least 0.8%, of xanthohumol of at least 3%, and of prenylated flavones selected from the group comprising 6-prenylnaringenin, 8-prenylnaringenin, and isoxanthohumol of at least 0.7%.

13. Use of an extract from hop as defined in claim 9 or 10 or a pharmaceutical preparation according to claim 11 or 12 for the preparation of a medicament for the prophylaxis and treatment of pathological diseases caused by a deficiency of oestrogens or a dysregulation of sex-hormone-related metabolism, particularly oestrogen metabolism, selected from the group consisting of climacteric complaints, benign prostate hypertrophy, osteoporosis, Alzheimer's disease and diseases of the cardiovascular system.

14. Use of an extract from hop as defined in claim 9 or 10 or a pharmaceutical preparation according to claim 11 or 12 for the preparation of a medicament for the prophylaxis against sex-hormone-dependent cancers.

15. Use according to claim 14, wherein the sex-hormone-dependent cancers are selected from the group consisting of breast cancer, carcinoma of the prostate, and carcinoma of the uterus.
